(19) United States
(12) Patent Application Publication
(10) Pub. No.: US 2019/0318664 A1
(43) Pub. Date: Oct. 17, 2019

(54) PATIENT AND MEDICAL RISK IDENTIFICATION ASSEMBLY

(71) Applicant: Bright Setorglo, Houston, TX (US)

(72) Inventor: Bright Setorglo, Houston, TX (US)

(21) Appl. No.: 15/954,690

(22) Filed: Apr. 17, 2018

(51) Int. Cl. G09F 3/00; G06K 7/14; A61B 90/90

(52) U.S. Cl. CPC G09F 3/005; A61B 90/90; G06K 7/1413

(57) ABSTRACT

A patient and medical risk identification assembly for enhancing patient care in a medical care environment includes a band that may be worn by a patient. A plurality of risk indicia is printed on the band to notify a medical care provider of the medical risk to the patient. A patient identifying indicia is printed on the band for identification of the patient. A plurality of covers is each removably coupled to the band to inhibit the risk indicia and the patient identifying indicia from being visible. Each of the covers is selectively removed from the band to reveal the associated ones of the risk indicia and the patient identifying indicia to notify the medical care provider of the medical risk to the patient.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to identification devices and more particularly pertains to a new identification device for enhancing patient care in a medical care environment.

### BRIEF SUMMARY OF THE INVENTION

An embodiment of the disclosure meets the needs presented above by generally comprising a band that may be worn by a patient. A plurality of risk indicia is printed on the band to notify a medical care provider of the medical risk to the patient. A patient identifying indicia is printed on the band for identification of the patient. A plurality of covers is each removably coupled to the band to inhibit the risk indicia and the patient identifying indicia from being visible. Each of the covers is selectively removed from the band to reveal the associated ones of the risk indicia and the patient identifying indicia to notify the medical care provider of the medical risk to the patient.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded perspective view of a patient and medical risk identification assembly according to an embodiment of the disclosure.

FIG. 2 is a bottom view of an embodiment of the disclosure.

FIG. 3 is a top view of an embodiment of the disclosure.

FIG. 4 is a cross sectional view taken along line 4-4 of FIG. 2 of an embodiment of the disclosure.

FIG. 5 is a perspective in-use view of an embodiment of the disclosure.

### DETAILED DESCRIPTION OF THE INVENTION

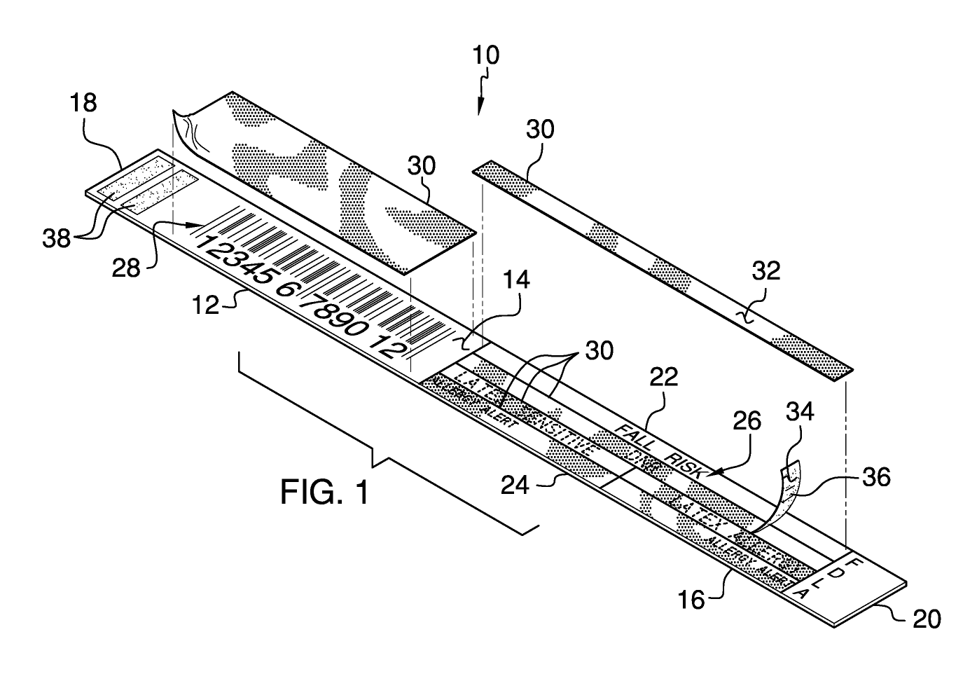

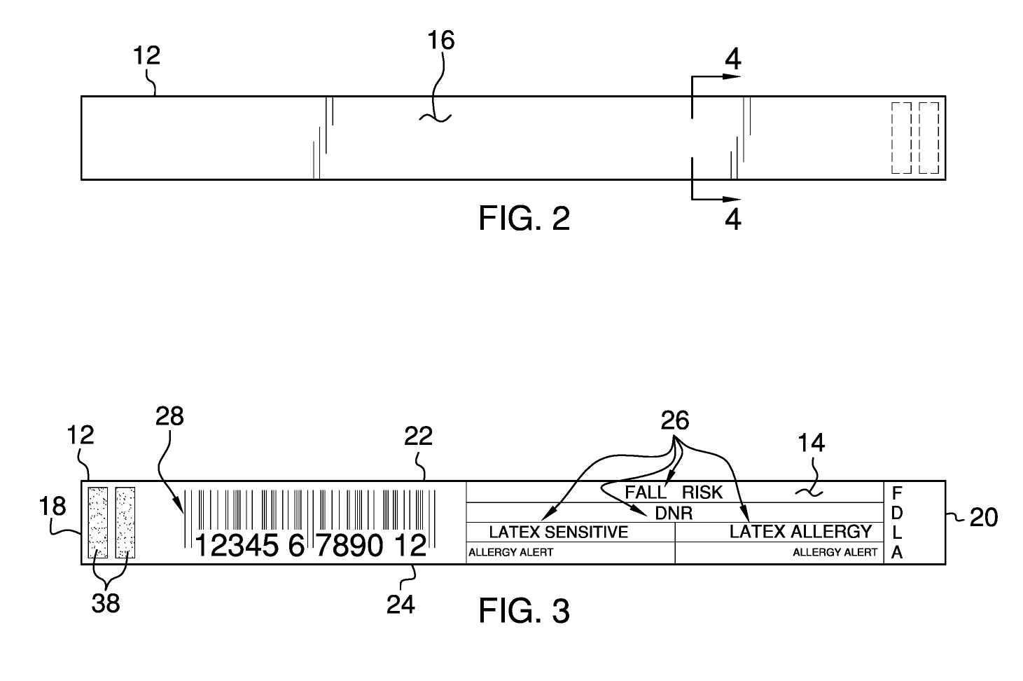

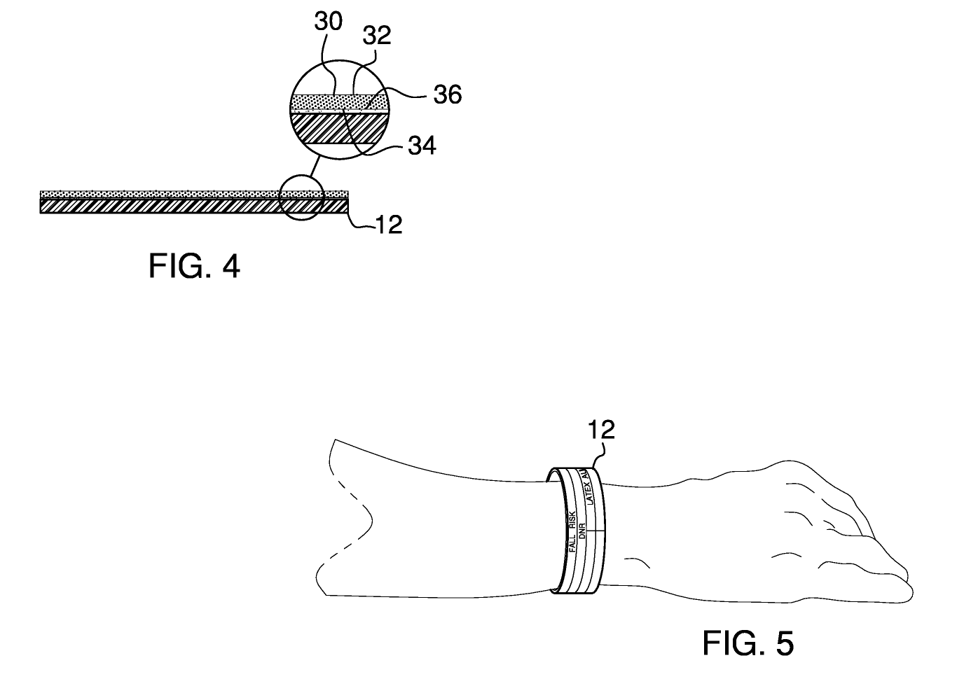

With reference now to the drawings, and in particular to FIGS. 1 through 5 thereof, a new identification device embodying the principles and concepts of an embodiment of the disclosure and generally designated by the reference numeral 10 will be described.

As best illustrated in FIGS. 1 through 5, the patient and medical risk identification assembly 10 generally comprises a band 12 that has a front surface 14, a back surface 16, a first lateral edge 18, a second lateral edge 20, a top edge 22 and a bottom edge 24. The band 12 is elongated between the first lateral edge 18 and the second lateral edge 20 and the band 12 is worn by a patient in a medical care environment. The medical care environment may be a hospital emergency room, a nursing home and any other medical care environment. Moreover, the band 12 may be comprised of a fluid impermeable material such as plastic or the like.

A plurality of risk indicia 26 is printed on the front surface 14 of the band 12 and each of the risk indicia 26 comprises a different a medical risk for notifying a medical care provider of the medical risk to the patient. The different medical risks may include a fall risk for elderly or physically disabled patients, DNR wishes of the patient, latex sensitivity of patient, latex allergy of the patient and any other allergy alerts pertaining to the patient. The risk indicia 26 may be arranged into a plurality of rows on the band 12, and each of the rows may be assigned a letter designator, such as “F’ for fall risk, “D” for DNR, “L” for latex allergies or latex sensitivity and “A” for other allergies.

A patient identifying indicia 28 is printed on the front surface 14 of the band 12. The patient identifying indicia 28 comprise a bar code wherein the bar code is associated with an identification of the patient. Thus, the bar code may be read with a bar code scanner utilized in the medical care environment for identifying patient information, including but not being limited to, the patient's name and medical history. Additionally, the bar code may be associated with data stored in an electronic database pertaining to the patient.

A plurality of covers 30 is provided and each of the covers 30 is removably coupled to the band 12. Each of the risk indicia 26 and the patient identifying indicia 28 has one of the covers 30 associated therewith. Moreover, each of the covers 30 is positioned over the associated one of the risk indicia 26 and the patient identifying indicia 28 and each of the covers 30 has a forward surface 32 and a rear surface 34. Each of the covers 30 is comprised of an opaque material to inhibit the risk indicia 26 and the patient identifying indicia 28 from being visible. Each of the covers 30 is selectively removed from the band 12 to reveal the associated ones of the risk indicia 26 and the patient identifying indicia 28 to notify the medical care provider of the medical risk to the patient. In this way the medical care provider can be informed at a glance each of the medical risks associated with the patient.

A plurality of adhesive layers 36 is provided and the rear surface 34 of each of the covers 30 has one of the adhesive layers 36 positioned thereon. Each of the adhesive layers 36 engages the front surface 14 of the band 12 such that each of the covers 30 removably covers 30 the associated one of the risk indicia 26 and the patient identifying indicia 28. Each of the adhesive layers 36 may comprise a non-residual adhesive or the like that has a bonding strength low enough to facilitate the covers 30 to be removed from the band 12.

A coupler 38 is positioned on the front surface 14 of the band 12 adjacent to the first lateral edge 18. The coupler 38 engages a back surface 16 of the band 12 adjacent to the second lateral edge 20 wherein the band 12 forms a closed loop shape. Moreover, the band 12 is worn on the wrist of a patient when the closed loop shape is formed. The coupler 38 comprises an adhesive and the adhesive associated with the coupler 38 may be an adhesive that has a bonding strength high enough to inhibit the closed loop formed by the band 12 from being broken. In this way the band 12 is inhibited from being inadvertently removed from the patient's wrist.

In use, the wrist band 12 is worn around the patient's wrist when the patient is admitted into the medical care environment. The cover associated with the patient identifying indicia is removed and the bar code is scanned with the bar code reader for accessing data associated with the patient identifying indicia 28. Additionally, selected ones of the covers 30 on the risk indicia 26 are removed to expose risk indicia 26 that correlate to medical risks of the patient. In this way the medical caregiver can quickly ascertain what medical risks are associated with the patient prior to performing medical care on the patient. Thus, the wrist band 12 helps to avoid common mistakes made in a medical care environment, such as exposing a latex sensitive patient to latex.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A patient and medical risk identification assembly comprising:
a band being configured to be worn by a patient;
a plurality of risk indicia being printed on said band wherein each of said risk indicia is configured to notify a medical care provider of said medical risk to the patient;
a patient identifying indicia being printed on said band for identification of the patient; and
a plurality of covers each being removably coupled to said band to inhibit said risk indicia and said patient identifying indicia from being visible, each of said covers being selectively removed from said band to reveal said associated ones of said risk indicia and said patient identifying indicia to notify the medical care provider of said medical risk to the patient.

2. The assembly according to claim 1, wherein said band has a front surface, a back surface, a first lateral edge, a second lateral edge, a top edge and a bottom edge, said band being elongated between said first lateral edge and said second lateral edge.

3. The assembly according to claim 2, wherein each of said risk indicia is printed on said front surface of said band, each of said risk indicia comprising a different said medical risk unique to each of said risk indicia.

4. The assembly according to claim 2, wherein said patient identifying indicia are printed on said front surface of said band, said patient identifying indicia comprising a bar code.

5. The assembly according to claim 1, wherein each of said risk indicia and said patient identifying indicia has one of said covers associated therewith wherein each of said covers is positioned over said associated one of said risk indicia and said patient identifying indicia.

6. The assembly according to claim 5, wherein each of said covers is comprised of an opaque material, each of said covers having a forward surface and a rear surface.

7. The assembly according to claim 6, further comprising a plurality of adhesive layers, said rear surface of each of said covers having one of said adhesive layers positioned thereon, each of said adhesive layers abutting said front surface of said band wherein each of said covers removably covers said associated one of said risk indicia and said patient identifying indicia.

8. The assembly according to claim 2, further comprising a coupler being positioned on said front surface of said band adjacent to said first lateral edge, said coupler engaging a back surface of said band adjacent to said second lateral edge wherein said band forms a closed loop shape, said band being configured to be worn on the wrist of a patient when said closed loop shape is formed.

9. The assembly according to claim 8, wherein said coupler comprises an adhesive.

10. A patient and medical risk identification assembly comprising:
a band having a front surface, a back surface, a first lateral edge, a second lateral edge, a top edge and a bottom edge, said band being elongated between said first lateral edge and said second lateral edge, said band being configured to be worn by a patient;
a plurality of risk indicia, each of said risk indicia being printed on said front surface of said band, each of said risk indicia comprising a different said medical risk unique to each of said risk indicia, each of said risk indicia being configured to notify a medical care provider of said medical risk to the patient;
a patient identifying indicia being printed on said front surface of said band, said patient identifying indicia comprising a bar code wherein said bar code is associated with an identification of the patient;
a plurality of covers being removably coupled to said band, each of said risk indicia and said patient identifying indicia having one of said covers associated therewith wherein each of said covers is positioned over said associated one of said risk indicia and said patient identifying indicia, each of said covers being comprised of an opaque material to inhibit said risk indicia and said patient identifying indicia from being visible, each of said covers being selectively removed from said band to reveal said associated ones of said risk indicia and said patient identifying indicia to notify the medical care provider of said medical risk to the patient, each of said covers having a forward surface and a rear surface;
a plurality of adhesive layers, said rear surface of each of said covers having one of said adhesive layers positioned thereon, each of said adhesive layers abutting said front surface of said band wherein each of said covers removably covers said associated one of said risk indicia and said patient identifying indicia; and
a coupler being positioned on said front surface of said band adjacent to said first lateral edge, said coupler engaging a back surface of said band adjacent to said second lateral edge wherein said band forms a closed loop shape, said band being configured to be worn on the wrist of a patient when said closed loop shape is formed, said coupler comprising an adhesive.
